(19) United States
(12) Patent Application Publication
(10) Pub. No.: US 2026/0274792 A1
(43) Pub. Date: Sep. 17, 2026

(54) POLYTHIOL COMPOSITIONS AND THEIR PROCESS OF PREPARATION FROM TERPENES OR FROM TERPENE DERIVATIVES

(71) Applicant: ARKEMA FRANCE, Puteaux (FR)

(72) Inventors: Pierre-Thomas SKOWRON, Lacq (FR); Sophie MENDES, Lacq (FR); Kévin TOURNEUR, Lacq (FR)

(73) Assignee: ARKEMA FRANCE, Puteaux (FR)

(21) Appl. No.: 19/469,862

(22) PCT Filed: Mar. 29, 2024
(86) PCT No.: PCT/FR2024/050410

§ 371 Date: Sep. 26, 2025

(30) Foreign Application Priority Data
Mar. 31, 2023 (FR) FR2303197

(51) Int. Cl. C07C 319/02; C07C 321/04

(52) U.S. Cl. CPC C07C 319/02; C07C 321/04

(57) ABSTRACT

The present invention relates to a process for the preparation of a polythiol from a terpene or from a terpene derivative, and also to polythiol compositions and novel polythiols.

The process comprises the following stages:
a) a terpene or a terpene derivative is reacted with a thiocarboxylic acid in the presence of oxygen (O₂) and of at least one organic solvent, so as to obtain a reaction medium comprising a polythioester and said at least one organic solvent;
b) a stage of deprotection of the polythioester obtained in stage a) is carried out, so as to obtain a polythiol;
   in which stage a) and stage b) are carried out in one-pot synthesis.

The present invention relates to a process for the preparation of polythiol compositions using terpenes or terpene derivatives as starting reactants, and also to the polythiol compositions obtainable by this process.

Polythiols are molecules of great industrial interest. They are used, for example, as crosslinking agents, in particular at low temperature.

There currently exist several synthetic routes for obtaining polythiols. Mention may be made, among the most widely used methods, of the reaction between polyols and mercaptoacids (described, for example, in Application US 2005153231). Although this reaction is easy and makes it possible to obtain varied polythiol structures, the products obtained generally exhibit low resistances to hydrolysis because of the significant presence of ester functions.

Alternatively, the direct addition of hydrogen sulfide to polyenes by acid or photochemical catalysis makes it possible to obtain molecules without hydrolysable functions. This additive is described in particular in Application WO 12018757. However, with this method, large amounts of sulfide-type compounds can be co-produced and, depending on the reactants used, conversion problems can arise. Thus, the molecules obtained can comprise numerous unconverted double bonds, which generates stability problems and lowers the overall content of —SH functions. In the case of a starting reactant of triene type, for example, this is reflected in particular by the presence of mono- and/or dithiols in a large amount in the composition obtained.

In point of fact, the control of and/or the reduction in the formation of these by-products, such as mono- and/or dithiols, is important depending on the targeted fields of application. This is because their content has an influence on the degree of crosslinking of the subsequently prepared materials, in particular thermosetting materials produced from a resin and from a hardener of polythiol type. It has thus been demonstrated that the degree of crosslinking influences the physical and viscoelastic properties of polymers, such as their density, their modulus, their limits of the elasticity range or their glass transition temperature (Tg). The Tg is conventionally determined by the DSC method for Differential Scanning Calorimetry or by dynamic mechanical analysis (DM(T)A).

These parameters are directly related to the behaviour of the materials, such as the hardness, the elasticity, the flexibility or also the tear strength. In particular, it is sought to obtain polymers having a high glass transition temperature, in order to obtain materials with a higher thermal resistance (that is to say, which retain their characteristics over a wider temperature range). There thus exists a need for a process for the industrial preparation of polythiols which makes it possible to control, indeed even to maximize, the conversion of the C═C double bonds into —SH functions. There also exists a need for a process for the preparation of polythiols which makes it possible to control, indeed even to reduce, the formation of by-products (for example mono- and/or dithiols in the case of the preparation of trithiols or also sulfides).

One technical solution consists in passing through polythioester intermediates: the C═C double bonds are converted into functions of R′—C(O)—S—R″ type, which are then deprotected in order to obtain the desired polythiols. However, these polythioester intermediates represent a great technical difficulty for industrial employment. They are generally very viscous, indeed even solid, compounds. They are thus difficult to be able to handle in order to be involved in the deprotection stage. They thus cause many practical problems at the industrial level and are in reality little used.

Moreover, these polythioesters are conventionally obtained by reaction of a polyene with a thiocarboxylic acid, in particular thioacetic acid. However, this reaction involves the use of a large excess of thiocarboxylic acid, which must be removed before the deprotection stage. Thus, additional stages of evaporation of this excess thiocarboxylic acid and/or of purification of the polythioesters are necessary in order to carry out the following deprotection stage.

Finally, the reactants used in this type of process are generally oil-derived hydrocarbons. In view of current environmental and climatic challenges, processes are desired which employ biobased and/or renewable starting materials.

There thus exists a need for an improved process for the preparation of polythiols, via polythioesters.

There also exists a need for an improved process for the preparation of polythiols, from biobased and/or renewable starting materials.

There exists a need for polythiol compositions, the content of —SH functions of which is controlled, indeed even maximized. The term “content of —SH functions” is understood to mean the ratio of the weight of all of the —SH functions/total weight of the composition.

There also exists a need for polythiol compositions obtained from biobased and/or renewable materials.

It is an objective of the present invention to provide an improved process for the preparation of polythiol compositions, from terpenes or from terpene derivatives, the industrial implementation of which is simplified.

It is an objective of the present invention to provide an improved process for the preparation of polythiol compositions, from terpenes or from terpene derivatives, by virtue of which the content of —SH functions is controlled, indeed even maximized.

It is also an objective of the present invention to provide improved polythiol compositions, in particular with a controlled, indeed even maximized, content of —SH functions.

It is also an objective of the present invention to provide polythiol compositions obtained from biobased and/or renewable starting materials, namely terpenes and terpene derivatives.

It is an objective of the present invention to provide polythiol compositions of use in the preparation of polymers, preferably of thermosetting polymers.

The present invention responds, in whole or in part, to the above objectives.

The present inventors have discovered, surprisingly, that it is possible to employ a “one-pot” process for the synthesis of polythiols from terpenes or from terpene derivatives. The term “one-pot process” is understood to mean in particular a process in which the synthesis intermediates (i.e. the polythioesters such as according to the invention) are not isolated from the reaction medium in order to carry out the following deprotection stage. In the context of the industrial synthesis of polythiols, such a one-pot process exhibits numerous advantages.

In particular, the stage of formation of the polythioester intermediates according to the invention (hereinafter stage a)) makes it possible to obtain a very good conversion (in particular between 90% and 100% conversion of the terpene or of the terpene derivative) while avoiding the use of a too great excess of thiocarboxylic acid. In fact, a large excess of thiocarboxylic acid is conventionally used in the processes of the prior art, which represents a loss for the process and generates a large amount of waste to be isolated and treated. Moreover, such an excess is not compatible with a “one-pot” process because it is necessary to remove it before the deprotection stage. The present invention makes it possible to avoid these drawbacks, which represents an economic advantage but also an environmental advantage.

Another advantage of the present invention is that the reaction medium comprising the polythioester intermediates obtained on conclusion of stage a) can be stirred and handled easily. It can in particular exist in the form of a liquid or of a suspension, which is viscous or slightly viscous. Difficulties of operability at the industrial level are thus avoided.

The reaction medium comprising the polythioester intermediates is also compatible with the deprotection stage (hereinafter stage b)), which represents a simplification of the process. Thus, stages a) and b) such as according to the invention are carried out as a “one-pot” reaction. The process is thus markedly improved because the intermediate stages of removal of the excess thiocarboxylic acid and/or of purification of the polythioester intermediates, such as extraction, recrystallization and/or distillation, are thus avoided.

In addition, terpenes and their derivatives may prove to be particularly useful as starting polyenes leading to polythiols. Terpenes and derivatives respond to many current environmental and climatic issues. They are generally biobased, that is to say result from renewable organic matter (biomass), of plant or animal origin. They also exhibit a great diversity of structures, which makes possible great versatility of uses.

The polythiol compositions capable of being obtained by the process according to the invention are novel and exhibit a controlled, indeed even maximized, —SH content. They are characterized in particular by a high

$$\frac{polythiol}{( x - 1 ) thiol ( s )}$$

ratio by weight as defined below. They are particularly suitable for the preparation of materials such as thermosetting plastics, from resins. Thus, the present invention makes it possible to obtain materials with superior properties. For example, polymers having a higher Tg, thus a superior thermal resistance, and/or superior compressive strength properties and/or a greater modulus and a greater elasticity range, can be obtained.

Thus, the present invention relates to a process for the preparation of a polythiol comprising the following stages:
a) a terpene or a terpene derivative is reacted with a thiocarboxylic acid in the presence of oxygen (O₂) and of at least one organic solvent, so as to obtain a reaction medium comprising a polythioester and said at least one organic solvent; and
b) a stage of deprotection of the polythioester obtained in stage a) is carried out, so as to obtain a polythiol;
in which stage a) and stage b) are carried out in one-pot synthesis.

The present invention relates to a polythiol composition A obtained from a terpene or from a terpene derivative having x C═C double bonds, said composition comprising:
the polythiol corresponding to said terpene or to said terpene derivative comprising x —SH functions; and
the thiol(s) corresponding to said terpene or to said terpene derivative comprising (x-1) —SH functions, with x being an integer greater than or equal to 3; and preferably in which the

$$\frac{polythiol}{( x - 1 ) thiol ( s )}$$

ratio by weight is between 1:1 and 50 000:1, preferably between 2:1 and 50 000:1.

The present invention also relates to a polythiol chosen from the trithiol obtained from dihydrofarnesene, the heptathiol obtained from isosqualene and the tetrathiol obtained from camphorene.

The term “alkyl” is understood to mean in particular a saturated, linear, branched or cyclic, hydrocarbon radical comprising from 1 to 10, preferably from 1 to 4, carbon atoms.

The term “aryl” is understood to mean in particular a cyclic (monocyclic, bicyclic or tricyclic) aromatic hydrocarbon radical comprising from 6 to 10 carbon atoms, preferably a phenyl or a naphthyl, more preferentially a phenyl.

The term “aralkyl” is understood to mean in particular an alkyl substituted by an aryl, for example benzyl.

### Terpenes

The term “terpenes” is understood to mean in particular linear, branched or cyclic hydrocarbon compounds consisting of isoprene repeat units (C₅C₈)ₙ, n being an integer of between 2 and 8, preferably of between 2 and 6. In particular, said terpenes comprise at least three C═C double bonds. Preferentially, said terpenes comprise 3, 4, 5 or 6 C═C double bonds.

Terpenes are often commercially available in the form of compositions comprising different isomers, the proportions of which can vary (in particular as a function of the process by which they are obtained). Such compositions come within the scope of the present invention and can be used directly as starting reactant.

The families of terpenes are conventionally categorized from the value of n (with the corresponding number of carbon atoms), according to the table below:

TABLE 1

| Value of n | 2 (C10) | 3 (C15) | 4 (C20) | 5 (C25) | 6 (C30) | 8 (C40) |
|---|---|---|---|---|---|---|
| Family | Monoterpenes | Sesquiterpenes | Diterpenes | Sesterterpenes | Triterpenes | Tetraterpenes |

In particular, the terpenes are chosen from monoterpenes, triterpenes and sesquiterpenes.

More particularly, the terpenes are chosen from linear or branched terpenes.

Mention may be made, among the preferred terpenes, of myrcene and farnesene.

### Myrcene:

Myrcene is a monoterpene. There exist various isomers of myrcene, including ocimene and alloocimene. In particular, mention may be made of α-myrcene, β-myrcene, cis-α-ocimene, trans-α-ocimene, cis-β-ocimene, trans-β-ocimene, 4-cis-6-cis-alloocimene, 4-cis-6-trans-alloocimene, 4-trans-6-cis-alloocimene and 4-trans-6-trans-alloocimene (cf. [FIG. 1]). Preferably, use is made of β-myrcene, which is the natural form (CAS No.: 123-35-3), of following formula:

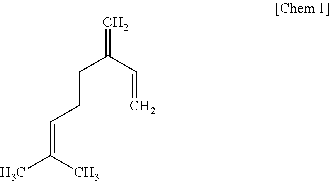

### Farnesene:

Farnesene is a sesquiterpene. It exists in the form of two isomers: α-farnesene (CAS No.: 502-61-4) and β-farnesene (CAS No.: 502-60-3) of following formulae:

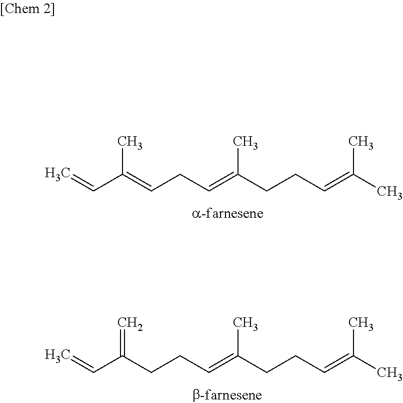

Mention may more particularly be made of cis-α-farnesene, trans-α-farnesene, cis-β-farnesene and trans-β-farnesene. β-Farnesene is preferred and more preferentially still trans-β-farnesene (CAS No.: 18794-84-8).

Use may be made, as other terpenes, of humulene (CAS 6753-98-6), elemene, germacrene, bisabolene, cembrene, casbene, zingiberene, camphorene and their isomers. In particular, mention may be made of the following isomers: α-elemene, β-elemene, γ-elemene, δ-elemene, germacrene A, germacrene B, germacrene C, germacrene D, germacrene E, α-bisabolene, β-bisabolene and γ-bisabolene.

### Terpene Derivatives:

The term “terpene derivatives” is understood in particular to mean compounds, the structure of which derives from that of the terpenes. They can result from the chemical transformation of the latter or can exist naturally. In particular, said terpene derivatives comprise at least three C═C double bonds, for example between 3 and 10 C═C double bonds.

Mention may be made of the following terpene derivatives.

### Hydrogenated Derivatives of Terpenes or Hydrogenated Terpenes:

The term “hydrogenated derivative of a terpene” is understood in particular to mean a compound of empirical formula (C₅ₙH_{8n+2z}), z being an integer at least equal to 1 and n being as defined above. Preferably, z is between 1 and 10, more preferentially between 1 and 3. Among the hydrogenated derivatives, squalene and dihydrofarnesene are particularly preferred.

Squalene (CAS No.: 111-02-4) is of the following formula:

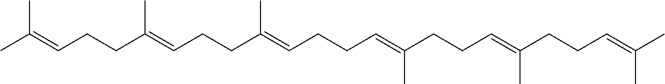 [SMILES: CC(C)=CCC/C(C)=C/CC/C(C)=C/CC/C=C(\C)CC/C=C(\C)CCC=C(C)C]

It has the empirical formula C₃₀H₅₀.

Dihydrofarnesene has the empirical formula C₁₅H₂₆. It can exist in the form of different isomers depending on the double bond of the hydrogenated farnesene and on the α or β isomer of the farnesene chosen. Dihydro-β-farnesene (and all of its isomers) is preferred.

More particularly, the following isomers of dihydrofarnesene can be used according to the present invention:

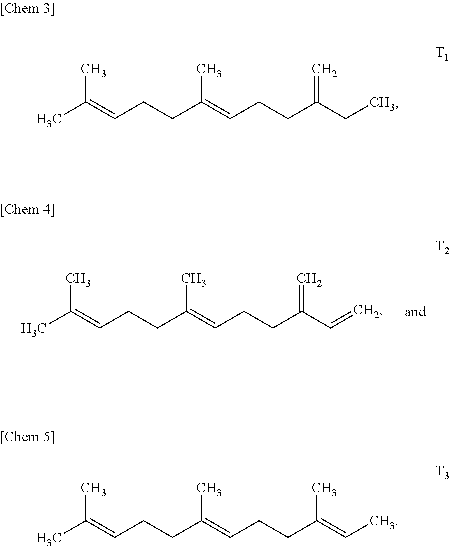

When a partial hydrogenation of the farnesene (C₁₅H₂₄) is carried out, it is also possible to obtain a composition comprising dihydrofarnesene and one or more other partially hydrogenated compounds chosen from:

tetrahydrofarnesene (C₁₅H₂₈) and hexahydrofarnesene (C₁₅H₃₀). Such a composition can be used as starting reactant in the context of the present invention: the dihydrofarnesene which it contains will give the corresponding trithiol according to the process according to the invention. Preferably, such a composition comprises at least 70% by weight of dihydrofarnesene, more preferably at least 80% by weight of dihydrofarnesene, with respect to the total weight of the composition. Preferably again, said composition comprises at least 85% by weight of dihydrofarnesene, with respect to the total by weight of unreacted farnesene, of all of the partially hydrogenated compounds resulting from farnesene and of farnesane present in the composition. In this type of composition, it is preferred particularly to use the β isomer of farnesene, in order to obtain dihydro-β-farnesene.

Thus, mention may preferentially be made of Myralene 10™ (CAS No.: 1581740-29-5), a composition obtained from the partial hydrogenation of β-farnesene and the process of preparation of which is given in Application WO 2016/064853. Myralene 10™ predominantly comprises dihydro-β-farnesene. Such a composition is entirely suitable as starting reactant according to the present invention.

### Terpenoids:

The term “terpenoids” is understood in particular to mean compounds, the structure of which derives from terpenes and which optionally comprise one or more heteroatom(s) (in particular oxygen and/or nitrogen, preferably oxygen) and/or one or more chemical function(s). For example, terpenoids can comprise at least one function chosen from alcohol, ketone, ether, ester or aldehyde functions.

They can in particular be of empirical formula (C₅ₙH_{8n-2y}), with n as defined above and y being an integer of between 1 and 4.

Preferably, the terpenoid is chosen from the group consisting of: cosmene, β-carotene, lycopene, farnesol, retinol, retinal, vitamin A, nerolidol, isomyrcenol and ipsdienol.

Oligomers of Terpenes and/or of Hydrogenated Derivatives of Terpenes and/or of Terpenoids:

The term “oligomer” corresponds in particular to an assembly of 2 to 10 terpenes and/or of hydrogenated derivatives of terpenes and/or of terpenoids as defined above, which are identical or different, preferably identical. Preferably, dimers and/or trimers of terpenes and/or of terpenoids are used. More preferentially, terpene dimers are used.

Mention may very particularly be made of the dimer of β-farnesene, called isosqualene, of empirical formula C₃₀H₄₈, and of following formula (for example as described in the document US 2011/0287988A1):

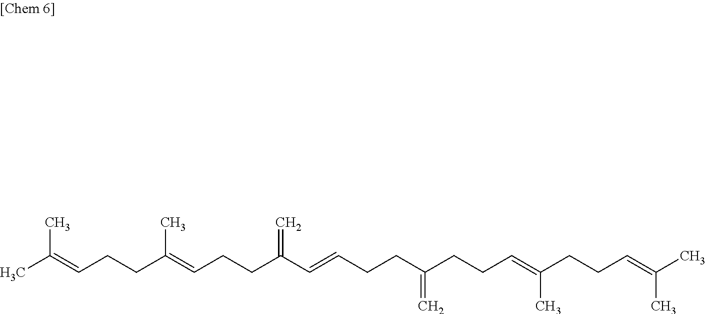 [SMILES: C=C(/C=C/CCC(=C)CC/C=C(\C)CCC=C(C)C)CC/C=C(\C)CCC=C(C)C]

The following isomers of isosqualene can also be used according to the present invention:

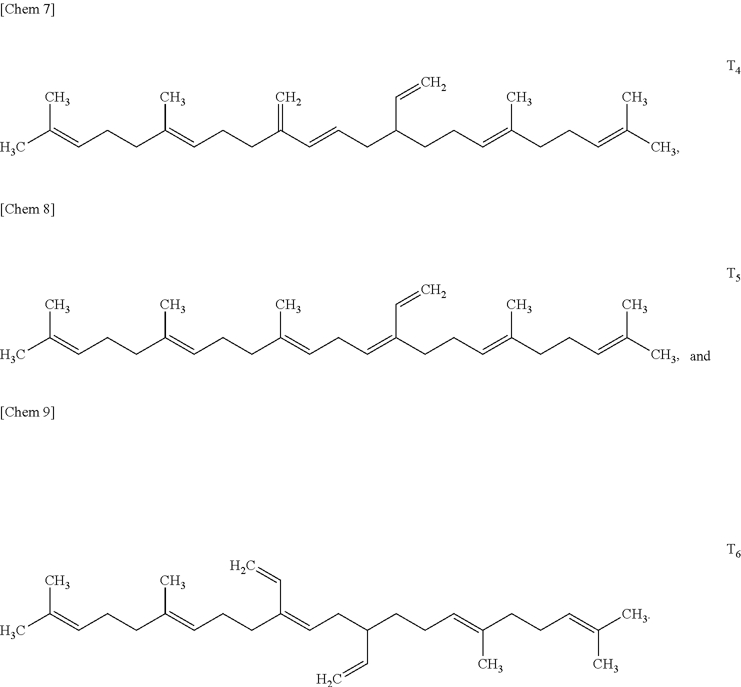

Thus, the term “terpenes and terpene derivatives” is preferentially understood to mean: terpenes, hydrogenated derivatives of terpenes, terpenoids and oligomers of terpenes and/or of hydrogenated derivatives of terpenes and/or of terpenoids.

Thus, said terpene or terpene derivative can be chosen from the group consisting of: myrcene, farnesene, humulene, elemene, germacrene, bisabolene, cosmene, cembrene, casbene, zingiberene, β-carotene, lycopene, camphorene, squalene, isosqualene, dihydrofarnesene, farnesol, retinol, retinal, vitamin A, nerolidol, isomyrcenol and ipsdienol. More preferentially, the terpene or terpene derivative is chosen from the group consisting of: myrcene, farnesene, squalene, isosqualene, humulene and dihydrofarnesene.

Such starting materials are naturally present in plants, in marine species or can be produced by fermentation, by genetically modified or non-genetically modified organisms and possibly using renewable carbon sources. They are also commercially available. For example, Amyris sells trans-β-farnesene under the name Biofene® and DRT sells myrcene.

### Polythiols

According to the invention, the term “polythiol” refers to the polythiol corresponding to the starting terpene or to the starting terpene derivative as defined above.

The term “corresponding to the starting terpene or to the starting terpene derivative” is understood to mean that the structure of the starting terpene or of the starting terpene derivative and of the polythiol obtained are identical, with the exception of the C═C double bonds, which have been converted into —SH functions (i.e. —CH—C(SH)—): for x C═C double bonds of the starting terpene or of the starting terpene derivative, x —SH functions are obtained. Also included under the term “polythiol” are the polythiols which are positional isomers of the double bonds.

The number of C═C double bonds contained in said terpene or terpene derivative is thus referred to hereinafter as “x”, x being an integer, preferably of greater than or equal to 3. Preferably, x is between 3 and 10, more preferentially between 3 and 7. The polythiols according to the invention can also be referred to as (x)thiols, with x as defined above.

### Polythioester Intermediates

The term “polythioester intermediate” or “polythioester” is understood to mean the polythioester corresponding to the starting terpene or to the starting terpene derivative. The term “corresponding to the starting terpene or to the starting terpene derivative” is understood to mean that the structure of the starting terpene or of the starting terpene derivative and of the polythioester obtained are identical, with the exception of the C═C double bonds, which have been converted into —CH—C(O)—S—R₁ functions (R₁ depends on the thiocarboxylic acid used; preferably, R₁ is a methyl): for x C═C double bonds, x thioester functions are obtained, with x as defined above. Also included under the term “polythioesters” are the polythioesters which are positional isomers of the double bonds of the starting terpene or terpene derivative.

### Process According to the Invention

### Stage a)

During stage a), a terpene or a terpene derivative as defined above is reacted with a thiocarboxylic acid in the presence of oxygen (O₂) and of at least one organic solvent, so as to obtain a reaction medium comprising a polythioester as defined above and said at least one organic solvent.

The reaction is as follows: R—CH═CH—R+R₁—C(O)—SH—>β—CH₂—CH(S—C(O)—R₁)—R

Stage a) is carried out in the presence of oxygen (O₂), acting here as initiator of the reaction.

Stage a) can thus be carried out in the presence of air, of depleted air (mixture of oxygen and nitrogen N₂) or of a mixture of oxygen and of another inert gas. The oxygen can be introduced into the reaction medium by any technique. The oxygen may or may not also be added throughout the duration of stage a).

In particular, the oxygen is bubbled into the reaction medium, preferably in the form of depleted air. For example, the depleted air is passed through a frit or a diffuser which dips into the reaction medium. Alternatively, oxygen can be bubbled into the reaction medium and nitrogen can be introduced into the gas phase of the reactor (that is to say, the headspace of the reactor).

The oxygen flow rate can be between 0.01 and 100 SI/h, preferably between 0.05 and 10 SI/h, more preferably between 0.05 and 5 SI/h, in particular between 0.05 and 2 SI/h (standard litres/h).

Stage a) is carried out in particular in the absence of any other initiator of the reaction, and more preferentially in the absence of AIBN (azobisisobutyronitrile) and/or in the absence of UV radiation.

Stage a) is also carried out in the presence of an organic solvent or of a mixture of organic solvents. A polar solvent or a mixture of polar solvents is very particularly chosen. The solvent(s) can be polar protic or polar aprotic solvent(s). Mention may be made, among the solvents which can be used, of: alcohols, ethers (preferably cyclic ethers and glycol ethers, such as, for example, glycol dialkyl ethers), organochlorinated solvents, carboxylic acids or their mixtures.

Preference is given to alcohols, in particular of following general formula (IV):

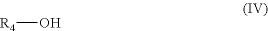

in which R₄ represents an alkyl as defined above. Preferably, the alcohol is chosen from the group consisting of: methanol, ethanol, isopropanol, n-propanol, n-butanol, butan-2-ol, isobutanol and tert-butanol, more preferably ethanol.

Preferably, the solvent is chosen from the group consisting of: tetrahydrofuran (THF), 2-methyltetrahydrofuran (Me-THF), dioxane, chloroform, acetic acid, methanol, ethanol, isopropanol, n-propanol, n-butanol, butan-2-ol, isobutanol, tert-butanol, dimethoxyethane (also referred to as glyme), diethoxyethane, dibutoxyethane and their mixtures, more preferentially ethanol.

The amount of solvent used is generally chosen as a function of the desired viscosity of the reaction medium. Complete or partial dissolution can be carried out by a person skilled in the art, depending on the targeted viscosity of the reaction medium. Preferentially, between 1 molar eq. and 50 molar eq., more preferably between 1 eq. and 20 eq., of solvent(s), with respect to the terpene or to the terpene derivative, is(are) used.

The solvent can be added from the beginning of stage a), completely or partially. It can be added in a one-off manner in one go, in several goes (semi-continuously) or gradually (continuously), during stage a).

The thiocarboxylic acid is preferably of following general formula (II):

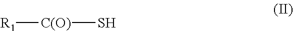

in which:
R₁ represents an alkyl radical, an aryl radical or an aralkyl radical as are defined above.

Preferably, R₁ is chosen from methyl, ethyl and benzyl.

Thioacetic acid, for which R₁ is a methyl, is very particularly preferred according to the invention (here-below also called ATA). For example, with thioacetic acid, a polythioacetate is obtained as polythioester intermediate.

According to one embodiment, the thiocarboxylic acid can be generated in situ (cf. the document U.S. Pat. No. 3,270,063, Thompson Chemical Co., 1963: “Methods of Making Primary Mercaptans”): thioacetic acid can be produced from acetic anhydride and hydrogen sulfide, in the presence of a catalyst.

Preferably, in order to carry out stage a), the thiocarboxylic acid and the solvent(s) are first introduced into the reactor, then the oxygen is introduced, for example by bubbling with air.

The terpene or derivative can subsequently be added to the reaction medium.

The temperature of stage a) can be between 5° C. and 80° C., preferably between 5° C. and 50° C., more particularly between 5° C. and 25° C., for example between 5° C. and 10° C. Stage a) is generally carried out at atmospheric pressure.

The thiocarboxylic acid/double bond of the terpene or terpene derivative molar ratio can be between 1 and 20, preferably between 1 and 10, for example between 1 and 5, more preferably between 1 and 3.

Stage a) makes it possible, starting from a terpene or from a terpene derivative, to form a polythioester intermediate as defined above.

The reaction medium obtained on conclusion of stage a) can thus comprise:
a polythioester intermediate as defined above;
the solvent or the mixture of solvents as defined above;
possibly by-products, such as (x-1)polythioesters; and
possibly one or more unreacted reactants.

The term “(x-1)polythioester” is understood to mean in particular a compound comprising x-1 thioester functions, with x being as defined above. It is a compound which has retained a C═C double bond (i.e. an unreacted C═C double bond).

The reaction medium can thus comprise between 10% and 85% by weight of polythioester intermediate, with respect to the whole of the reaction medium.

The reaction medium can comprise between 15% and 90% by weight of solvent(s), with respect to the whole of the reaction medium.

### Stage b)

Stage b) of deprotection of the polythioester intermediate obtained in stage a) makes it possible to obtain a polythiol. It can be carried out by any means known to a person skilled in the art. As stage a) and stage b) are carried out in a one-pot synthesis according to the invention, it is understood that the reaction medium comprising the polythioester obtained on conclusion of stage a) is retained in order to carry out the deprotection stage b). Thus, stages a) and b) are carried out in the presence of the same solvent (or mixture of solvents). It is possible to add more of said solvent (or said mixture of solvents) during stage b). In particular, the process according to the invention does not comprise a stage of separation and/or of extraction and/or of washing of the (organic) phase which comprises the polythioester between stages a) and b). In particular, no intermediate stage of purification of the polythioester is carried out. More particularly, no stage of recrystallization and/or of distillation of the polythioester is carried out. The deprotection b) can be carried out by conventional methods: using a base or an acid, a catalyst of Dy(OTf)₃ type (cf. Liang et al., Asian J. Org. Chem., 10.1002/ajoc.201700481) or a compound of quaternary ammonium cyanide salt type (cf. U.S. Pat. No. 7,173,156).

Preferably, the deprotection b) is a basic deprotection, preferably in the presence of an alcohol as defined above. It is generally carried out by addition of an alkaline hydroxide, preferably NaOH or KOH. The addition can be carried out dropwise.

The deprotection stage b) can also be an acidic deprotection, preferably in the presence of an alcohol as defined above. It can be carried out with hydrochloric acid, methanesulfonic acid or anhydrous methanesulfonic acid. When the deprotection is acidic, it is preferable to use an alcohol as defined above as solvent.

### Sulfonic Acid

The sulfonic acid is preferably an organosulfonic acid which is optionally anhydrous.

The sulfonic acid can be of following general formula (III):

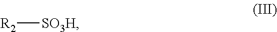

where R₂ represents:
   an alkyl radical, preferably as defined above, optionally substituted, in all or in part, by one or more identical or different halogen atoms, or
   an aryl radical, preferably as defined above, optionally substituted by a saturated, linear or branched, hydrocarbon chain comprising from 1 to 4 carbon atoms.

The halogen atom can be chosen from fluorine, chlorine and bromine. In particular, said alkyl can be perhalogenated, more particularly perfluorinated.

Preferably, the sulfonic acid is an alkanesulfonic acid which is optionally anhydrous (in the above formula, R₂ is an alkyl).

Thus, the sulfonic acids (and also their anhydrous forms) can be chosen from: methanesulfonic acid, ethanesulfonic acid, n-propanesulfonic acid, isopropanesulfonic acid, n-butanesulfonic acid, isobutanesulfonic acid, sec-butanesulfonic acid, tert-butanesulfonic acid, trifluoromethanesulfonic acid, para-toluenesulfonic acid, benzenesulfonic acid and the mixtures of two or more of them in all proportions.

According to a very particularly preferred embodiment, the sulfonic acid used in the context of the present invention is methanesulfonic acid (MSA) or anhydrous methanesulfonic acid (AMSA).

Said sulfonic acid may or may not be supported. Preferably, it is not supported.

When it is supported, it is possible, for example, to use sulfonated resins of styrene-divinylbenzene copolymer type, for example Amberlyst® 15 resin or Nafion®.

For example, between 0.1 and 10 eq. of acid are used for a polythioester.

For example, between 3 and 60 eq. (molar equivalent), preferably between 3 and 20 eq., of alcohol are used for a polythioester.

The deprotection stage b) can be carried out at a temperature of between 10° C. and 100° C., preferably between 25° C. and 80° C., more preferably between 40° C. and 80° C. It is generally carried out at atmospheric pressure.

Stages a) and b) can be carried out in the same reactor. For example, a batch reactor can be used.

Subsequent stages of conventional recovery and/or of conventional purification of the polythiol recovered on conclusion of stage b) can be carried out, depending on the desired degree of purity. For example, when the deprotection is carried out by the addition of a base, the reaction medium can subsequently be acidified and conversely, when the deprotection is carried out by the addition of an acid, the reaction medium can be basified.

The organic phase resulting therefrom and comprising the various thiols (in particular the polythiol and the (x-1)thiols) can subsequently be extracted and optionally concentrated. Thus and in particular, the polythiol obtained can be in the form of a polythiol composition as mentioned below.

### Compositions According to the Invention

When a polythiol is prepared from a terpene or from a terpene derivative having x C═C double bonds, the conversion of these double bonds into —SH functions is not generally complete and by-products can be formed at each of the various stages, whatever the process used. According to the invention, the term “polythiol” thus refers to the polythiol corresponding to the starting terpene or to the starting terpene derivative and comprising x —SH functions. In this case, the conversion of the starting x C═C double bonds into —SH functions is complete. According to the invention, the term “(x-1)thiol” refers to a thiol corresponding to the starting terpene or to the starting terpene derivative and comprising (x-1) —SH functions. The term “corresponding to the starting terpene or to the starting terpene derivative” is understood to mean that the structure of the starting terpene or of the starting terpene derivative and of the (x-1)thiol obtained are identical, with the exception of the x C═C double bonds, which have been converted into (x-1) —SH functions. In this case, the conversion of the C═C double bonds into —SH functions has not been complete: an —SH function is missing.

The C═C double bond not converted into an —SH function can in particular be:
still in the form of a C═C double bond; or
in the form of a thioester function which has not been deprotected.

There can thus exist different structures of (x-1)thiols, but they are here combined under this general name characterizing their number of —SH functions (unless specifically mentioned otherwise). Also included are the (x-1)thiols which are positional isomers of the double bonds For example, during stage a), (x-1)thioesters can be formed. In this case, for x starting C═C double bonds, only (x-1) C═C double bonds react with the thiocarboxylic acid to form (x-1) thioester functions.

Moreover, during stage b), it is also possible for the deprotection not to be complete.

Thus, it is possible to form, according to the process according to the invention:
(x-1)thiols from the (x-1)thioesters formed in stage a); and/or
(x-1)thiols from the polythioesters which are not completely deprotected.

It is thus possible to obtain a polythiol composition resulting from a terpene or from a terpene derivative having x C═C double bonds, comprising:
the polythiol corresponding to said terpene or to said terpene derivative comprising x —SH functions; and
the thiol(s) corresponding to said terpene or to said terpene derivative comprising (x-1) —SH functions;
   with x as defined above.

Such a composition can optionally comprise other by-products or impurities (for example monothiols).

In particular, it is possible to obtain a trithiol composition from a terpene or from a terpene derivative having three C═C double bonds, said composition comprising:
the trithiol corresponding to said terpene; and
the dithiol(s) corresponding to said terpene.

In particular, it is possible to obtain a tetrathiol composition from a terpene or from a terpene derivative having four C═C double bonds, said composition comprising:
the tetrathiol corresponding to said terpene or to said terpene derivative; and
the trithiol(s) corresponding to said terpene or to said terpene derivative.

Thus, the present invention relates to a polythiol composition A obtained from a terpene or from a terpene derivative having x C═C double bonds, said composition comprising:
the polythiol corresponding to said terpene or to said terpene derivative comprising x —SH functions; and
the thiol(s) corresponding to said terpene or to said terpene derivative comprising (x-1) —SH functions, with x as defined above.

In particular, said composition A comprises at least 50% by weight, preferably at least 60% by weight, for example at least 70% by weight, more preferably at least 80% by weight, more preferentially at least 90% by weight, for example at least 95% by weight, of said polythiol, with respect to the total weight of the composition A.

In particular, said composition A comprises less than 40% by weight, preferably less than 30% by weight, more preferably less than 25% by weight, of said (x-1)thiol(s), with respect to the total weight of said composition A.

Preferably, the

$$\frac{polythiol}{( x - 1 ) thiol ( s )}$$

ratio by weight of the composition A is between 1:1 and 20 000:1, for example between 1:1 and 10 000:1, preferably between 1:1 and 1000:1, more preferably between 1:1 and 100:1, and more preferentially between 1:1 and 10:1, for example between 2:1 and 10:1.

Said ratio by weight is the ratio by weight: [polythiol corresponding to said terpene or terpene derivative comprising x —SH functions]/[thiol(s) corresponding to said terpene or terpene derivative comprising (x-1) —SH functions].

In a very particularly preferred way, said composition A is obtained from a terpene or a terpene derivative chosen from myrcene, farnesene, squalene, isosqualene, humulene and dihydrofarnesene.

### POLYTHIOLS ACCORDING TO THE INVENTION

The present invention also relates to the trithiol resulting from dihydrofarnesene, the heptathiol resulting from isosqualene and the tetrathiol resulting from camphorene (i.e. the trithiol corresponding to dihydrofarnesene, the heptathiol corresponding to isosqualene and the tetrathiol corresponding to camphorene).

Preferably, said trithiol is the trithiol of dihydro-β-farnesene. The trithiol of dihydrofarnesene can in particular be in the form of one of its following positional isomers:

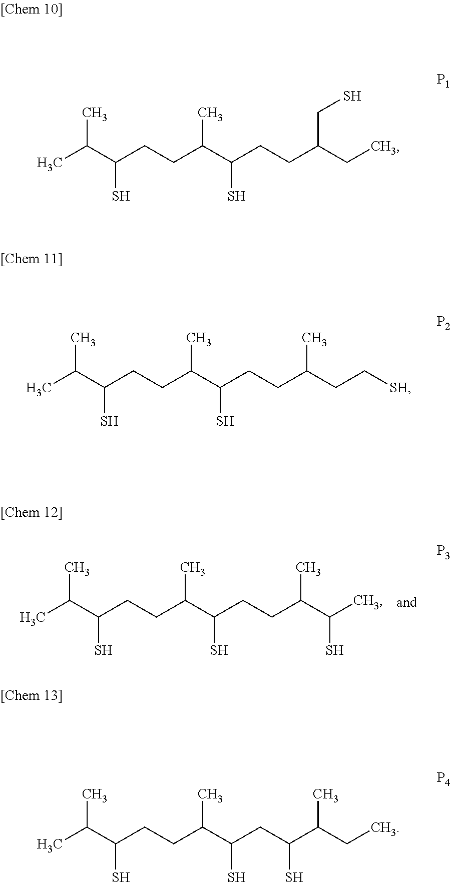 [SMILES: CCC(CS)CCC(S)C(C)CCC(S)C(C)C ; CC(CCS)CCC(S)C(C)CCC(S)C(C)C ; CC(C)C(S)CCC(C)C(S)CCC(C)C(C)S ; CCC(C)C(S)CC(S)C(C)CCC(S)C(C)C]

Said trithiol of dihydrofarnesene can be obtained from a starting composition comprising at least 70% by weight of dihydrofarnesene, more preferably at least 80% by weight of dihydrofarnesene, with respect to the total weight of the composition. Preferably, said composition comprises at least 85% by weight of dihydrofarnesene, with respect to the total by weight of farnesene, of all of the partially hydrogenated compounds resulting from the hydrogenation of farnesene and of farnesane present in said composition.

More particularly, said composition is a trithiol of dihydro-β-farnesene composition obtained from Myralene 10™.

The heptathiol of isosqualene can in particular be in the form of one of its following positional isomers:

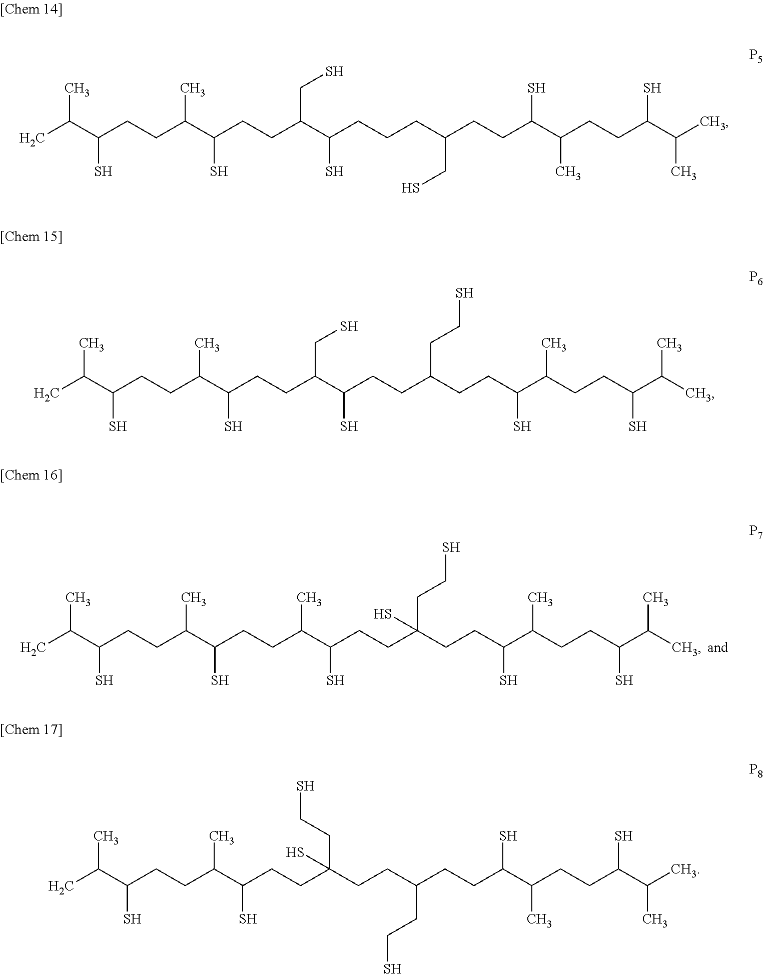 [SMILES: CC(C)C(S)CCC(C)C(S)CCC(CS)CCCC(S)C(CS)CCC(S)C(C)CCC(S)C(C)C ; CC(C)C(S)CCC(C)C(S)CCC(CCS)CCC(S)C(CS)CCC(S)C(C)CCC(S)C(C)C ; CC(C)C(S)CCC(C)C(S)CCC(C)C(S)CCC(S)(CCS)CCC(S)C(C)CCC(S)C(C)C ; "and" ; CC(C)C(S)CCC(C)C(S)CCC(CCS)CCC(S)(CCS)CCC(S)C(C)CCC(S)C(C)C]

The present invention also relates to polythiols resulting from β-carotene, lycopene, farnesol, retinol, retinal, vitamin A, nerolidol, isomyrcenol and ipsdienol.

These compounds are novel and form part of the present invention.

The present invention also relates to the compositions as defined above and capable of being obtained, obtained or directly obtained by the process according to the invention. Likewise for the polythiols as defined above, which can be capable of being obtained, obtained or directly obtained by the process according to the invention.

### DESCRIPTION OF THE FIGURES

FIG. 1: Isomers of myrcene

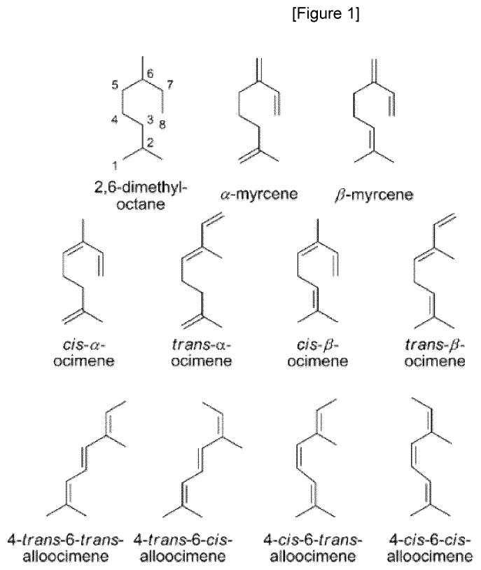

It is understood that, unless a specific isomer is mentioned, the name of a compound comprises all of its possible positional isomers.

The examples which follow are given by way of illustration and do not limit the present invention.

### EXAMPLES

### Example 1: Trithiol of Dihydro-β-Farnesene Obtained from Myralene 10™, Process According to the Invention

### Stage a):

48.7 g (0.64 mol) of TAA are introduced into a 250-ml jacketed reactor. The medium is placed under stirring at 5° C. Air is bubbled into the reaction medium via a frit at a flow rate of approximately 0.4 SI/h and nitrogen is passed into the headspace of the reactor at a flow rate of approximately 4 SI/h.

40 g (0.19 mol) of Myralene 10™ are subsequently added dropwise via a peristaltic pump over approximately 27 min. Once the addition is complete, 35.7 g (0.77 mol) of EtOH are added to the reaction medium.

The reaction medium is kept stirred at 5° C. overnight.

A GC/FID analysis shows complete conversion of the Myralene 10.

The air supply is cut off.

### Stage b) of Basic Deprotection:

26.8 g (0.58 mol) of EtOH are then added to the reaction medium. The reaction medium is subsequently degassed with nitrogen for 1 h, is then cooled to approximately 10° C. and 103 g (0.64 mol) of a 25% sodium hydroxide solution, degassed beforehand, are added in 38 min via a dropping funnel. The reaction medium is left stirring under nitrogen at 10° C. overnight and then at 25° C. for 6 h.

A GC/FID analysis shows complete conversion of the trithioacetate.

### Recovery Stage:

The reaction medium is subsequently cooled to 20° C. and 117 g (0.64 mol) of 20% HCl (degassed beforehand with nitrogen) are then added dropwise to the reaction medium via a peristaltic pump. The trithiol phase is withdrawn. The aqueous phase is extracted three times with 16.5 g (0.19 mol) of dichloromethane.

The organic phases are combined, then washed four times with 7 g (0.39 mol) of water and then concentrated on a rotary evaporator.

A composition comprising 82.29% by weight of the trithiol of dihydro-β-farnesene and 9.71% by weight of the dithiol of dihydro-β-farnesene is obtained (with respect to the total weight of the composition).

The

$$\frac{polythiol}{( x - 1 ) thiol ( s )}$$

ratio by weight is 82.29:9.71, i.e. 8.5:1.

### Example 2: Trithiol of Myrcene, Process According to the Invention

### Stage a):

64.5 g (0.85 mol) of TAA are introduced into a 250-ml jacketed reactor. The medium is placed under stirring at 5° C. and then 5.9 g (0.13 mol) of EtOH are rapidly added. Air is bubbled into the reaction medium via a frit at a flow rate of approximately 0.4 SI/h and nitrogen is passed into the headspace of the reactor at a flow rate of approximately 4 SI/h.

35 g (0.26 mol) of myrcene are subsequently added dropwise via a peristaltic pump over approximately 22 min. Once the addition is complete, 23.7 g (0.51 mol) of EtOH are added to the reaction medium.

The reaction medium is kept stirred at 5° C. overnight.

A GC/FID analysis shows complete conversion of the myrcene.

The air supply is cut off.

### Stage b) of Basic Deprotection:

82.9 g (1.80 mol) of EtOH are then added to the reaction medium. The reaction medium is subsequently degassed with nitrogen for 1 h, is then brought to approximately 30° C. and 73.7 g (0.85 mol) of a 46% sodium hydroxide solution, degassed beforehand, are added via a dropping funnel. The reaction medium is left stirring under nitrogen at 40° C. for 21 h.

A GC/FID analysis shows complete conversion of the trithioacetate.

### Recovery Stage:

The reaction medium is subsequently cooled to 20° C. and 154.6 g (0.85 mol) of 20% HCl (degassed beforehand with nitrogen) are then added dropwise to the reaction medium via a peristaltic pump. The trithiol phase is withdrawn. The aqueous phase is extracted three times with 21.8 g (0.26 mol) of dichloromethane.

The organic phases are combined, then washed four times with 9.2 g (0.51 mol) of water and then concentrated on a rotary evaporator.

A composition comprising 50.56% by weight of the trithiol of myrcene and 23.6% by weight of the dithiol of myrcene is obtained (with respect to the total weight of the composition). The

$$\frac{polythiol}{( x - 1 ) thiol ( s )}$$

ratio by weight is 50.56:23.6, i.e. 2.1:1.

### Example 3: Tetrathiol Obtained from Farnesene, Process According to the Invention

### Stage a):

163.9 g (2.15 mol) of TAA are introduced into a 1-litre jacketed reactor. The medium is placed under stirring at 5° C. and then 23.0 g (0.50 mol) of EtOH are rapidly added. Air is bubbled into the reaction medium via a frit at a flow rate of approximately 0.4 SI/h and nitrogen is passed into the headspace of the reactor at a flow rate of approximately 4 SI/h.

100 g (0.49 mol) of farnesene, sold under the brand name Biofene®, are subsequently added dropwise via a peristaltic pump over approximately 47 min. Once the addition is complete, 90.1 g (1.96 mol) of EtOH are added to the reaction medium.

The reaction medium is kept stirred at 5° C. overnight.

A GC/FID analysis shows complete conversion of the farnesene and its reaction intermediates.

The air supply is cut off.

### Stage b) of Basic Deprotection:

The reaction medium is subsequently degassed with nitrogen for 1 h, is then cooled to approximately 10° C. and 187.2 g (2.15 mol) of a 46% sodium hydroxide solution, degassed beforehand, are added in 35 min via a dropping funnel. The reaction medium is left stirring under nitrogen at 25° C. for 19 h.

A GC/FID analysis shows complete conversion of the tetrathioacetate.

### Recovery Stage:

The reaction medium is subsequently cooled to 20° C. and 294.4 g (1.61 mol) of 20% HCl (degassed beforehand with nitrogen) are then added dropwise to the reaction medium via a peristaltic pump. The tetrathiol phase is withdrawn. The aqueous phase is extracted three times with 41.6 g (0.49 mol) of dichloromethane.

The organic phases are combined, then washed four times with 17.6 g (0.98 mol) of water and then concentrated on a rotary evaporator.

A composition comprising 61.24% by weight of the tetrathiol of farnesene and 25.77% by weight of the trithiol of farnesene is obtained (with respect to the total weight of the composition).

The

$$\frac{polythiol}{( x - 1 ) thiol ( s )}$$

ratio by weight is 61.24:25.77, i.e. 2.4:1.

1. Process for the preparation of a polythiol comprising the following stages:
a) a terpene or a terpene derivative is reacted with a thiocarboxylic acid in the presence of oxygen (O₂) and of at least one organic solvent, so as to obtain a reaction medium comprising a polythioester and said at least one organic solvent; and
b) a stage of deprotection of the polythioester obtained in stage a) is carried out, so as to obtain a polythiol;
in which stage a) and stage b) are carried out in one-pot synthesis.

2. Preparation process according to claim 1, in which stage b) of deprotection is a basic deprotection, preferably carried out by addition of an alkaline hydroxide.

3. Process according to claim 1, in which stage b) of deprotection is an acidic deprotection, preferably in the presence of an alcohol.

4. Preparation process according to claim 1, in which said organic solvent is chosen from the group consisting of: alcohols, ethers, organochlorinated solvents, carboxylic acids and their mixtures.

5. Preparation process according to claim 1, in which the organic solvent is chosen from the alcohols of following general formula (IV):
R₄—OH (IV)
in which R₄ represents a saturated, linear, branched or cyclic, hydrocarbon radical comprising from 1 to 10, preferably from 1 to 4, carbon atoms.

6. Preparation process according to claim 1, in which the thiocarboxylic acid is thioacetic acid.

7. Preparation process according to claim 1, in which said terpene or terpene derivative is chosen from the group consisting of myrcene, farnesene, squalene, isosqualene, humulene and dihydrofarnesene.

8. Polythiol composition A obtained from a terpene or from a terpene derivative having x C═C double bonds, said composition comprising:
the polythiol corresponding to said terpene or to said terpene derivative comprising x —SH functions; and
the thiol(s) corresponding to said terpene or to said terpene derivative comprising (x-1) —SH functions;
with x being an integer greater than or equal to 3.

9. Polythiol composition A according to claim 8, in which the
$$\frac{polythiol}{( x - 1 ) thiol ( s )}$$
ratio by
weight is between 1:1 and 50 000:1, preferably between 2:1 and 50 000:1.

10. Polythiol composition A according to claim 8, in which said terpene or terpene derivative is chosen from the group consisting of myrcene, farnesene, squalene, isosqualene, humulene and dihydrofarnesene.

11. Polythiol chosen from the trithiol obtained from dihydrofarnesene, the heptathiol obtained from isosqualene and the tetrathiol obtained from camphorene.

12. Polythiol according to claim 11, having one of the following formulae: [chemical structure]
